(19) United States
(12) Patent Application Publication
(10) Pub. No.: US 2022/0168411 A1
(43) Pub. Date: Jun. 2, 2022

(54) PSORALEN-INACTIVATED CORONAVIRUS VACCINE AND METHOD OF PREPARATION

(71) Applicant: The United States of American as Represented by the Secretary of the Navy, Silver Spring, MD (US)

(72) Inventors: Kevin R Porter, Boyds, MD (US); Maya Willams, Silver Spring, MD (US); Raviprakash Kanakatte, Clarksville, MD (US); Dawn Weir, Kensington, MD (US); Daniel Ewing, Laurel, MD (US); Appavu K Sundaram, (US)

(73) Assignee: The United States of American as Represented by the Secretary of the Navy, Silver Spring, MD (US)

(21) Appl. No.: 17/539,667

(22) Filed: Dec. 1, 2021

Related U.S. Application Data
(60) Provisional application No. 63/119,866, filed on Dec. 1, 2020.

(51) Int. Cl. A61K 39/12

(52) U.S. Cl. CPC A61K 39/12; A61K 2039/55572

(57) ABSTRACT

The invention reported here relates to a method for preparation of inactivated SARS-CoV-2 vaccine by exposing the virus (SARS-CoV-2) to a predetermined concentration of an inactivating psoralen compound, and a preselected intensity of ultraviolet A (UVA) radiation for a preselected time period long enough to render the virus inactive but short enough to prevent degradation of its antigenic characteristics.

### CROSS-REFERENCE TO RELATED APPLICATIONS

This application claims priority to U.S. Provisional Application No. 63/119,866 filed on Dec. 01, 2020, which is hereby incorporated by reference.

### STATEMENT REGARDING FEDERALLY SPONSORED RESEARCH OR DEVELOPMENT

This invention was made with Government support under contracts DHP RDT&E supplemental COVID funding that were awarded by the Defense Health Program, U.S. Department of Defense, and the Joint Staff. The Government has certain rights in the invention.

### FIELD OF INVENTION

The present invention relates to the development of inactivated coronavirus vaccines. Specifically, the invention relates to the method of preparation of psoralen-inactivated SARS-CoV-2 vaccine.

### BACKGROUND OF INVENTION

Since the 2000s, three coronaviruses have crossed species barriers to infect humans and cause acute severe respiratory illness: severe acute respiratory syndrome coronavirus (SARS-CoV) in 2003, Middle East respiratory syndrome coronavirus (MERS-CoV) in 2012, and SARS-CoV-2 in 2019 [1, 2]. SARS-CoV-2 infections in humans emerged in December 2019 and has since spread throughout the world causing the COVID-19 (Corona virus disease of 2019) pandemic. As of 26 Aug. 2021 there have been more than 218 million confirmed cases of COVID-19 and more than 4.5 million COVID-19 related deaths worldwide [3]. Pfizer and BioNTech developed an mRNA vaccine (COMIRNATY) against COVID-19 that was formally approved by U.S. Food and Drug Administration (FDA) on 23 Aug. 2021. Besides this FDA approved vaccine, there are several Emergency Use Authorized (EUA) COVID-19 vaccines that are currently in use. However, there is a concern regarding the efficacy of these COVID-19 vaccines against the Variants of Concern (VOC) such as beta (B.1.351), delta (B.1.617.2) that have emerged and are now circulating worldwide. Currently there are no anti-viral drugs to effectively treat SARS-Co-V-2 infections. Therefore, development of a safe and broadly protective vaccine against SARS-CoV-2 VOCs is a global priority owing to their high rate of disease transmission and high number of hospitalizations and deaths worldwide.

Most of the COVID-19 vaccines that are currently in use and in preclinical and clinical stages are designed to elicit immune responses to the SARS-CoV-2 Spike protein [4, 5]. While these vaccines show short-term protective efficacy against SARS-CoV-2 infection, it is not clear if they will exhibit long term efficacy [6]. Besides, emergence of SARS-CoV-2 VOCs with spike protein mutations (such as the currently circulating delta Omicron variants) raises a big concern regarding the efficacy of these vaccines against such variants. Therefore, development of a whole virus inactivated vaccine targeting several viral antigens including the spike protein, membrane protein and nucleocapsid protein is expected to provide broader protection against several VOCs. Although the traditional, formalin-inactivated whole virus vaccine approach is a promising platform for developing a vaccine, formaldehyde treatment has been shown to cause intermolecular cross links between proteins leading to altered conformational changes and antigenic epitopes [7]. At least three whole inactivated virus vaccines have been authorized for emergency use: CoronoVac (manufactured by Sinovac, China), BBIBP-CorV (manufactured by Sinopharm, China), and Covaxin (manufactured by Bharat Biotech, India) [8, 9]. Phase III clinical trial results for Coronovac indicated a wide range of protective efficacy, from 50% efficacy in Brazil to more than 90% efficacy in Turkey. Protection efficacy of BBIBP-CorV has been suggested to be between 70-80%, although the phase III results haven't been published yet. Although phase III trial data for Covaxin is not published yet, it has been suggested to provide high levels of protective efficacy. All three inactivated vaccines are prepared by β-propiolactone inactivation of the whole virus. Wide range of protective efficacies observed for these vaccines may be due to altered conformation of antigenic epitopes caused by interactions with β-propiolactone [10, 11]. Psoralen is a furanocoumarin that intercalates with nucleic acids and upon exposure to long wave ultraviolet radiation (UVA) leads to inter-strand cross-links by covalently binding to pyrimidine bases [12-14]. Therefore, psoralen inactivation serves to inactivate the virus at the nucleic acid level, with presumably better preservation of the antigenic epitopes of the surface proteins. Virus treated with psoralen and long wavelength ultra violet (UV) irradiation can't replicate due to the crosslinks within the virus nucleic acid and hence are rendered inactive. Previously, we prepared highly purified monovalent and tetravalent dengue vaccines inactivated using psoralen-(DENV PsIV) or formalin (DENV FIV) and evaluated their immunogenicity in mice and nonhuman primates [15]. Significantly higher neutralizing antibody titers for each dengue serotype were observed both in mice and nonhuman primates vaccinated with the tetravalent DENV PsIVs compared to those vaccinated with the tetravalent DENV FIVs. This supports the use of psoralen inactivation for development of an inactivated SARS-CoV-2 vaccine that is broadly protective against SARS-CoV-2 VOCs.

### BRIEF SUMMARY OF INVENTION

This invention relates to preparation of a psoralen-inactivated whole virus vaccine against coronavirus, specifically a psoralen-inactivated SARS-CoV-2 vaccine. The inventive subject matter is a method of SARS-CoV-2 inactivation using psoralen to prepare non-replicative whole virus immunogen, and the method of purification of psoralen-inactivated SARS-CoV-2 to make highly purified psoralen-inactivated SARS-CoV-2 vaccine. According to the present invention, SARS-CoV-2 whole virus inactivated vaccines are prepared by treating coronavirus such as SARS-CoV-2 with a psoralen compound, such as 4′-aminomethyl trioxsalen (AMT), followed by exposure to UVA light for a period of time. Psoralen-inactivated SARS-CoV-2 vaccine prepared per method described in present invention may comprise one or more inactivated variants/strains of SARS-CoV-2. The conditions of inactivation used in this invention ensures inactivation of SARS-CoV-2, without loss of immunogenicity. The method of inactivation of SARS-CoV-2 used in this invention can also be used for preparing whole virus inactivated vaccines against other coronaviruses, such as SARS-CoV-2, MERS-CoV, human coronavirus OC43, human coronavirus HKU1 or SARS-CoV.

A further aspect of the invention includes preparation of highly purified psoralen-inactivated SARS-CoV-2 vaccine using chromatographic method.

### BRIEF DESCRIPTION OF THE DRAWINGS

FIG. 1 illustrates the steps of SARS-CoV-2 culture preparation, psoralen-inactivation of SARS-CoV-2 and purification of psoralen-inactivated SARS-CoV-2.

FIG. 2 shows Microneut₈₀ data (day 56 sera) from BALB/C mice vaccinated with SARS-CoV-2 PsIV vaccine with different adjuvant. Circles represent individual mice, and horizontal bars represent the geometric mean for each group. * indicates significant differences (p≤0.05) between the adjuvant and PsIV group vs the corresponding adjuvant alone group.

FIG. 3 shows Microneut₈₀ data (day 71 sera) from BALB/C mice vaccinated with SARS-CoV-2 PsIV vaccine with different adjuvant. Circles represent individual mice, and horizontal bars represent the geometric mean for each group. * indicates significant differences (p≤0.05) between the adjuvant and PsIV group vs the corresponding adjuvant alone group.

FIG. 4 Total IgG endpoint titers to SARS-CoV-2 spike protein (S), Receptor binding Domain (RBD) and nucleocapsid protein from day 71 mice sera (14 days after receiving the booster dose of the vaccines). Each symbol represents one mouse, and horizontal lines represent geometric mean for each group. * indicates significant differences between adjuvant and PsIV group vs corresponding adjuvant alone group.

FIG. 5 shows IFN-γ, IL-2, and IL-4 responses to antigen stimulation. Mouse spleenocytes were stimulated with peptide pools representing whole length spike (divided into two pools S1 and S2), nucleocapsid (N), membrane (M), and envelope (E) proteins. Antigen-specific responses to the S peptide pools, the N peptide pool, and the M and E peptide pools are shown in black, red, and blue, respectively. Data are presented as SFUs per 1×10⁶ cells. Each symbol represents one mouse, and horizontal lines represent geometric mean for each group. * indicates significantly differences (p≤0.05) between the adjuvant and PsIV groups vs the corresponding adjuvant alone group.

FIG. 6 shows Microneut₈₀ data (day 51 sera) from NHPs vaccinated with SARS-CoV-2 PsIV vaccine with Advax-2 adjuvant. Circles represent individual animal, and horizontal bars represent the geometric mean for each group.

### DETAILED DESCRIPTION OF THE INVENTION

Coronavirus vaccines are prepared by inactivation of live corona virus, such as SARS-CoV-2 virus, in a medium containing an amount of an inactivating psoralen sufficient to inactivate the virus upon subsequent irradiation with ultraviolet radiation of predetermined intensity (UV). Degradation of the antigenic characteristics of the live virus is reduced or eliminated by carefully selecting psoralen(s) of a pre-determined concentration and exposing the virus to only minimum intensity and duration of UV necessary to inactivate the virus.

Psoralens may be used in the inactivation process include psoralen and substituted psoralens, in which the substituent may be alkyl, particularly having from one to three carbon atoms, e.g., methyl; alkoxy, particularly having from one to three carbon atoms, e.g., methoxy; and substituted alkyl having from one to six, more usually from one to three carbon atoms and from one to two heteroatoms, which may be oxy, particularly hydroxy or alkoxy having from one to three carbon atoms, e.g., hydroxy methyl and methoxy methyl, or amino, including mono- and dialkyl amino or aminoalkyl, having a total of from zero to six carbon atoms, e.g., aminomethyl. There will be from 1 to 5, usually from 2 to 4 substituent, which will normally be at the 4,5,8,4′ and 5′ positions, particularly at the 4′ position. Illustrative compounds include 5-methoxypsoralen; 8-methoxypsoralen (8-MOP); 4,5′, 8-trimethylpsoralen (TMP); 4′-hydroxymethyl-4,5′,8-trimethylpsoralen (HMT); 4′-aminomethyl-4,5′,8-trimethyl psoralen (AMT); 4-methylpsoralen; 4,4′-dimethylpsoralen; 4,5′-dimethylpsoralen; 4′,8-dimethylpsoralen; and 4′-methoxymethyl-4,5′,8-trimethylpsoralen. Of particular interest are AMT4,5′, TMP and 8-MOP.

Different psoralens may be used individually or in combination. The psoralens may be present in amounts ranging from 1-200 μg/ml, preferably about 50 μg/ml. In carrying out the invention, the psoralen(s) are combined with the viral suspension, conveniently a viral suspension in an aqueous buffered medium, such as those used for storage.

Although viral inactivation according to the present invention will normally be carried out in an inactivation medium as just described, it may be desirable to introduce psoralens to the virus by addition to a cell culture medium in which the virus is grown. The inactivation is then carried out by separating the live viral particles from the culture medium, and exposing the particles to ultraviolet light in the inactivation medium which may or may not contain additional psoralens.

When employing psoralens with limited aqueous solubility, typically below about 50 g/ml, it has been found useful to add an organic solvent, such as dimethyl sulfoxide (DMSO), ethanol, glycerol, polyethylene glycol (PEG) or polypropylene glycol, to the aqueous treatment solution. For psoralens having limited solubility, such as 8-MOP, TMP, and AMT, adding a small amount of such organic solvents to the aqueous composition, typically in the range from about 1 to 25% by weight, more typically from about 2 to 10% by weight, the solubility of the psoralen can be increased to about 50 μg/ml, or higher. Such increased psoralen concentration may permit the use of shorter irradiation times. Also, inactivation of particularly recalcitrant microorganisms may be facilitated without having to increase the length or intensity of ultraviolet exposure. The addition of an organic solvent may be necessary for inactivation of some viruses with particular furocoumarins. The ability to employ less rigorous inactivation conditions is of great benefit in preserving the antigenicity of the virus during inactivation.

The time of UV irradiation will vary depending upon the light intensity, the concentration of the psoralen, the concentration of the virus, and the manner of irradiation of the virus receives, where the intensity of the irradiation may vary in the medium. The time of irradiation will be inversely proportional to the light intensity. The total energy applied should be no less than 1.45 joule and preferably from approximately 1.5 Joules to 10 Joules. UV irradiation usually last at least about 5 minutes and no more than 60 minutes, generally ranging from about 5 to 10 minutes.

The light, which is employed, will generally have a wavelength in the range from about 300 nm to 400 nm. Usually, an ultraviolet light source will be employed together with a filter for removing UVB light. The intensity will generally range from about 150 μW/cm2 to about 1500 μW/cm2, although in some cases, it may be higher.

Prior to treatment with ultraviolet light, the virus furocoumarin solution is placed upon a bed of ice. The temperature for the irradiation is preferably under 25° C. During irradiation, the medium may be maintained still, stirred or circulated. The solution may be either continuously irradiated or be subjected to alternating periods of irradiation and non-irradiation. The circulation may be in a closed loop system or in a single pass system ensuring that the entire sample has been exposed to irradiation.

It may be desirable to remove the unexpended psoralen and/or its photodegradation products from the irradiation mixture. This can be readily accomplished by one of several standard laboratory procedures such as chromatography or dialysis across an appropriately sized membrane or through an appropriately sized hollow fiber system after completion of the irradiation. Alternatively, one could use affinity methods for one or more of the low molecular weight materials to be removed.

The inactivated virus may then be formulated in a variety of ways for use as a vaccine. The concentration of the virus will generally be from about 1×10⁵ to 1×10¹² pfu/mL, and preferably about 10⁸-10¹⁰ PFU/ml, as determined prior to inactivation.

In an embodiment of the present invention, SARS-CoV-2 whole virus inactivated vaccines are prepared by treating SARS-CoV-2 with a psoralen compound, specifically 4′-aminomethyl trioxsalen (AMT), followed by exposure to UVA light for a certain time to render the virus non-replicative. Psoralen-inactivated SARS-CoV-2 vaccine comprises inactivated SARS-CoV-2 of one or more variants/strains of SARS-CoV-2. The conditions of inactivation used in this invention ensures inactivation of SARS-CoV-2 without loss of immunogenicity. The method of inactivation of SARS-CoV-2 used in this invention can also be used for preparing vaccines against other coronaviruses. Whole virus inactivated vaccines are prepared by inactivation of live SARS-CoV-2 virus in a medium containing an amount of psoralen (AMT) sufficient to inactivate the virus upon subsequent exposure to UVA light (at 365 nm). Antigenic characteristics of the live virus is preserved by selecting the minimal amount of psoralen (pre-determined concentration) required and exposing the virus to a minimal UVA energy and time required to inactivate the virus. Specifically, 30 μg of AMT per 1 mL of virus was added and the mixture was exposed to UVA light until 1.5 joules of energy (approximately 5 minutes) was applied to the AMT-virus mixture. Highly purified viral vaccines may be prepared by subjecting the psoralen-inactivated SARS-CoV-2 vaccine to chromatographic purification using resins such as Cellufine MAX DexS VirS resin. Suitable vaccine formulation for the inoculation of humans and animals may be prepared by combining the highly purified inactivated virus with an adjuvant, such as an alum or Advax™-2 adjuvant at an appropriate amount to elicit immune responses. Other vaccine adjuvants may include ASO4 (monophosphoryl lipid A), MF59 (oil-in-water emulsion containing squalene) and CpG1018 (cytosine phosphoguanine motifs).The present invention is suitable for preparing psoralen-inactivated SARS-CoV-2 vaccines comprised of one or more variants. Psoralen-inactivation of each variant of SARS-CoV-2 may be carried out individually and combined together. The psoralen-inactivated SARS-CoV-2 vaccine prepared according to this invention may protect against disease by one or more SARS-CoV-2 VOCs.

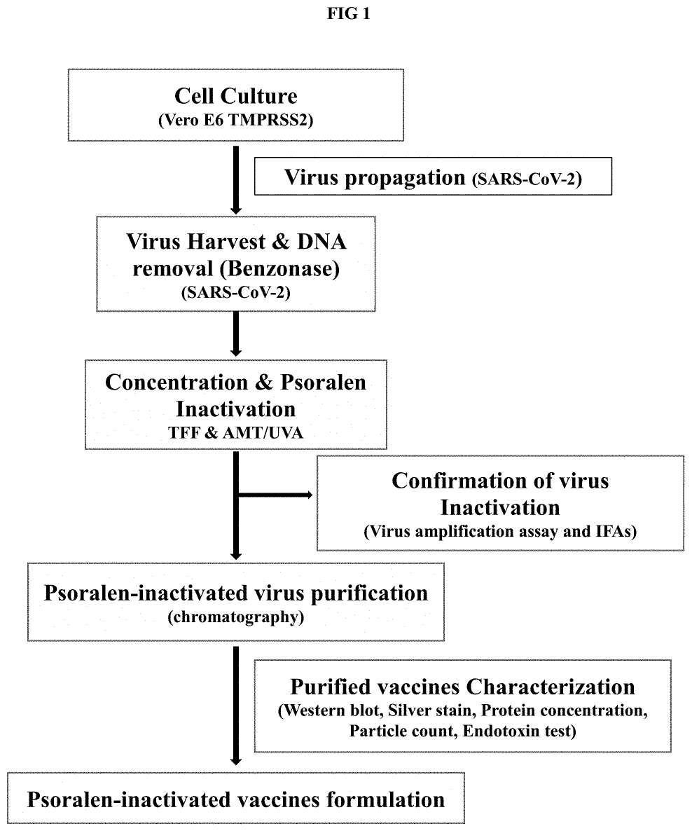

According to FIG. 1, sufficient amounts of virus required for inactivation may be obtained by growing the seed virus in an appropriate mammalian cell culture. The seed virus for propagation may be obtained from a vendor or by isolation from an infected host. Appropriate mammalian cell cultures include primary or secondary cell cultures derived from mammalian tissues or established cell lines such as Vero E6, Vero 81 and Vero E6 TMPRSS2 cells. The cell cultures are grown to 90-95% confluency and then infected with the virus at a low multiplicity of infection (MOI), preferably at about 0.001.

Psoralen used for coronavirus inactivation may include psoralen and substituted psoralen compounds. Substituents in psoralen may include alkyl (methyl), alkoxy, and substituted alkyl groups. Examples of different psoralen derivatives that may be used for coronaviruses inactivation include 4′-aminomethy trioxsalen (AMT), 8-methoxypsoralen (8-MOP), and 4,5′,8-trimethylpsoralen (TMP). Different psoralen compounds may be used for inactivation either individually or in combination for inactivation of different coronaviruses. After psoralen inactivation of the virus, the psoralen degradation products and the excess psoralen may be removed from the inactivated virus by size exclusion chromatography or multimodal chromatography such as Capto Core 700 column.

This psoralen-inactivated whole virus immunogen was evaluated in mice for its ability to elicit neutralizing Abs against SARS-CoV-2.

### EXAMPLE 1

### Psoralen-Inactivation of SARS-COV-2

SARS-CoV-2 strain nCoV/USA-WA1/2020 was propagated in Vero cell cultures and harvested by centrifugation at 3000× g for 15 minutes. 500-2000 mL of the culture supernatant containing SARS-CoV-2 was then treated with benzonase (an enzyme degrading free nucleic acids) to remove host cell nucleic acids in the culture supernatant and the volume was reduced to 50-200 mL (concentrating) using 100K MWCO membrane filter cassettes. The concentrated SARS-CoV-2 virus preparation was mixed with psoralen derivative 4′-aminomethyl trioxsalen (AMT), at 30 μg of AMT per 1 mL of virus, and the resulting mixture was then treated with long wavelength UV light (λ=365 nm) for 5 minutes (total energy applied=1445400 μjoules). Complete inactivation of psoralen and UVA treated SARS-CoV-2 virus was confirmed by its inability to grow in permissive cells by a two passage virus amplification test.

Briefly, 50 μL aliquots of the inactivated virus was used to infect cultured cells in duplicate. After incubation at 37° C. for 5-8 days, cells and culture supernatants were examined for the presence of SARS-CoV-2 antigens by indirect immunofluorescence assay and plaque assay respectively. Negative results for these analyses (indicating the absence of SARS-CoV-2 antigens) confirmed that the virus has been completely inactivated. The supernatant from this culture was then used for infecting fresh cells for a second round of amplification and testing. Negative results (indicating the absence of virus specific antigens) in the second test further confirmed the complete psoralen-inactivation of SARS-CoV-2.

TABLE 1
Conditions for psoralen-inactivation of SARS-CoV-2.

| Amount of Psoralen per mL | Total long wavelength UV |
|---|---|
| of SARS-CoV-2 virus | energy delivered (at 4818 μJoules/s) |
| 30 μg/mL | 1445400 μjoules (5 minutes) |

### EXAMPLE 2

### Purification of SARS-CoV-2 PsIV

Psoralen-inactivated SARS-CoV-2 vaccine (SARS-CoV-2 PsIV) prepared according to Example 1, was purified by sucrose gradient centrifugation. Presence of SARS-CoV-2 antigen in the pure SARS-CoV-2 PsIV fraction was confirmed by western blot using SARS-CoV-2 specific anti-spike protein and anti-nucleoprotein antibodies. Purity of SARS-CoV-2 PsIV was assessed by gel electrophoresis followed by silver staining. SARS-CoV-2 PsIV titer was determined using Virocyt 2.0.

In another embodiment of the invention, SARS-CoV-2 PsIV is purified by chromatographic methods using Cellufine Max Dex VirS resin. Briefly, psoralen-inactivated SARS-CoV-2 in 10 mM tris buffer containing 150 mM sodium chloride is passed through a 25 mL Max Dex VirS column at a flow rate of 0.5 mL per minute, followed by washing with 2 column volumes of 10 mM tris buffer containing150 mM sodium chloride. SARS-CoV-2 PsIV bound to the column resin is then eluted using 10 mM tris-HCl buffer containing 500 mM sodium chloride. Fractions containing SARS-CoV-2 PsIV in this elution buffer, identified by Western blot analysis using an anti-SARS-CoV-2 spike protein antibody, are then pooled together as purified SARS-CoV-2 PsIV, then passed through a buffer exchange column with PBS and stored at −80° C. until further use. Presence of SARS-CoV-2 antigens in the purified SARS-CoV-2 PsIV fraction was confirmed by western blot using SARS-CoV-2 specific anti-spike protein and anti-nucleoprotein antibodies.

### EXAMPLE 3

### Evaluation of Immunogenicity of SARS-CoV-2 PsIV in Balb/c Mice

SARS-CoV-2 PsIV vaccine was evaluated with alum or Advax-2 adjuvant, as illustrated in Table 2. Groups of 4 mice were immunized with different vaccines/adjuvants by intradermal inoculation of 50 μL of vaccine candidates. Animals in group 1 received alum (adjuvant) on days 1 and 29. Animals in group2 received advax-2 (adjuvant) on days 1 and 29. Different titers of SARS-CoV-2 PsIV vaccines with either alum or advax-2 (as indicated in the table) were administered intradermally on days 1 and 29 to groups 3, 4, 5 and 6.

TABLE 2
Vaccination groups (4 animals/group) and doses

| Groups | Adjuvant + Vaccine | Dose |
|---|---|---|
| 1 | Alum Control | PBS + alum |
| 2 | Advax -2 control | PBS + Advax-2 |
| 3 | Alum + SARS-CoV-2 PsIV | 10⁵ particles of SARS-CoV-2 |
| | (low dose) | PsIV |
| 4 | Advax-2 + SARS-CoV-2 PsIV | 10⁵ particles of SARS-CoV-2 |
| | (low dose) | PsIV |
| 5 | Alum + SARS-CoV-2 PsIV | 10⁷ particles of SARS-CoV-2 |
| | (high dose) | PsIV |
| 6 | Advax-2 + SARS-CoV-2 PsIV | 10⁷ particles of SARS-CoV-2 |
| | (high dose) | PsIV |

Blood was drawn from all animals on days 0, 28 and 56. All the animals were boosted with the respective vaccines/adjuvants on day 57 and euthanized on day 71 for harvesting spleens (to measure the T-cell responses). Blood was collected from all the animals on day 71, before euthanasia.

Anti-SARS-CoV-2 neutralizing antibody in serum was assayed using a microneutralization test. Two hundred TCID₅₀ of SARS-CoV-2 virus was incubated with two-fold dilutions of serum samples for 60 minutes in a 96 well plate. Vero81 cells (2>10\hat{ }4) were then added to each well and incubated at 37° C. for 84 h. After 84 h, the cells were fixed and SARS-CoV-2 was measured by quantitating spike protein using SARS-CoV-2 specific anti-S antibody in a standard ELISA format. The highest serum dilution that resulted in ≥80% reduction in absorbance when compared to control was determined to be the 80% microneutralization titer (MN₈₀).T cell assays were also conducted for each vaccination group. On day 72 (14 days after administering the booster doses) mouse spleens were harvested and placed in a 6-well plate containing RPMI 1640 media with 10% fetal bovine serum, 1% penicillin-streptomycin mixture, and 50 μM 2-mercaptoethanol. A small incision was made in the splenic capsule to allow for the diffusion of splenocytes into the media, which was facilitated by the application of gentle pressure using a 1 mL syringe. The media was gently mixed to break up any cell aggregates and transferred to a 50 mL centrifuge tube through a 70 μm cell strainer. The centrifuge tubes were spun at 1600 rpm for 8 min at 4° C., and the resulting pellet was suspended in 40 mL of chilled phosphate-buffered saline (PBS). The tubes were centrifuged again at 1280 rpm for 8 min at 4° C., and the pellet was resuspended in PBS before being counted for recovery and viability. Fresh spleen cells were used for IFN-γ and IL-2 ELISPOT assays, and the remaining cells were frozen at a concentration of 1×10⁷ cells/mL in fetal bovine serum containing 10% dimethyl sulfoxide (DMSO). Frozen cells were thawed and used for the IL-4 ELISPOT assay.

### EXAMPLE 4

### Evaluation of Immunogenicity of SARS-CoV-2 PsIV in Nonhuman Primates

SARS-CoV-2 PsIV vaccine in combination with Advax-2 adjuvant was evaluated in nonhuman primates at different doses, as illustrated in Table 3. Two doses of SARS-CoV-2 PsIV vaccines were administered by intramuscular injection (IM) with PharmaJet Stratus needle-less injector on days 0 and 30. Animals in the control group (group 1) received advax-2 adjuvant) on days 0 and 30. Blood was drawn from all animals on days 0, 30 and 51 for measuring the presence of neutralizing antibodies. Efficacy of SARS-CoV-2 PsIV vaccine in NHPs will be evaluated by challenging the vaccinated animals with SARS-CoV-2 delta variant, sixty days after administering the second dose of SARS-CoV-2 PsIV and observing the animals for clinical symptoms and lung viral loads for up to 14 days. Four animals from each group were challenged with SARS-CoV-2 delta strain on day 90 by intranasal instillation of 1×10⁵ PFU of SARS-CoV-2 delta strain (current SARS-CoV-2 VOC) in 0.5 mL (0.25 mL per nostril). After the virus challenge, nasal and throat swabs were collected from each animal on every other day for up to 14 days. Bronchalveolar lavage samples from the virus-challenged animals are also collected on days 3, 6 and 9 post-challenge. These samples will be used for determining viral loads in the respiratory tracts and the lungs of the challenged animals.

TABLE 3
Animal groups (n = 8) and SARS-CoV-2
PsIV doses for evaluation in NHPs

| | | Amount of | |
|---|---|---|---|
| | | SARS-CoV-2 | |
| | | spike protein | |
| Groups | Vaccine dose (particles/dose) | per dose | |
| 1 | Advax -2 control (0) | 0 | |
| 2 | SARS-CoV-2 PsIV 10⁸ particles/dose | 0.008 | μg/dose |
| 3 | SARS-CoV-2 PsIV 10⁹ particles/dose | 0.08 | μg/dose |
| 4 | SARS-CoV-2 PsIV 10¹⁰ particles/dose | 0.8 | μg/dose |
| 5 | SARS-CoV-2 PsIV 5 × 10¹⁰ particles/dose | 4 | μg/dose |

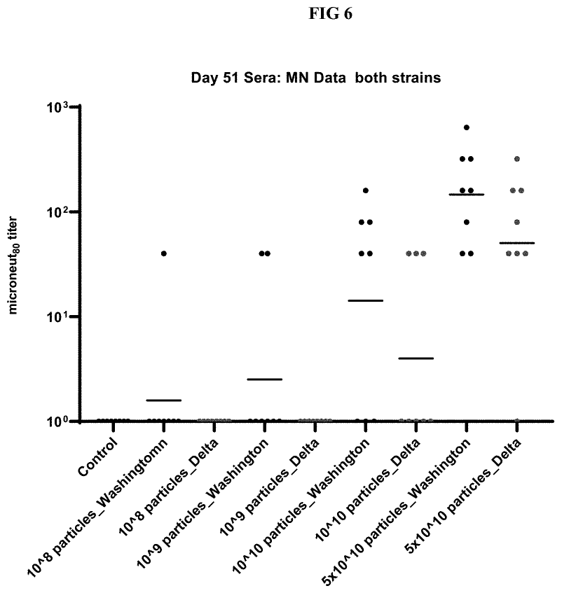

Anti-SARS-CoV-2 neutralizing antibody in serum was assayed using a microneutralization test against both the Washington strain and the delta variant (B.1.617.2). Two hundred TCID₅₀ of SARS-CoV-2 virus was incubated with two-fold dilutions of day 51 serum samples (3 weeks after the administration of the second dose of the vaccine) for 60 minutes in a 96 well plate. Vero81 cells (2×10\hat{ }4) were then added to each well and incubated at 37° C. for 84 h. After 84 h, the cells were fixed and SARS-CoV-2 was measured by quantitating spike protein using SARS-CoV-2 specific anti-S antibody in a standard ELISA format. The highest serum dilution that resulted in ≥80% reduction in absorbance when compared to control was determined to be the 80% microneutralization titer (MN₈₀). Three weeks after administration of the second dose of psoralen-inactivated SARS-CoV-2 vaccines, a robust dose-dependent neutralizing antibody responses in NHPs was observed (FIG. 6). Higher doses of SARS-CoV-2 PsIV (10¹⁰ and 5×10¹⁰ particles/dose) elicited neutralizing antibody responses against both the Washington strain and the delta strain. These results suggest that SARS-CoV-2 PsIV is capable eliciting robust immune responses against SARS-CoV-2 variants of concern.

Data analysis was performed using GraphPad prism 8.3.1. One way ANOVA with Bonferroni's multiple comparison test was performed for group-wise analysis. Statistical significance is indicated as *P<0.05, **P<0.01, ***P<0.001, and ****P<0.0001.

### Results

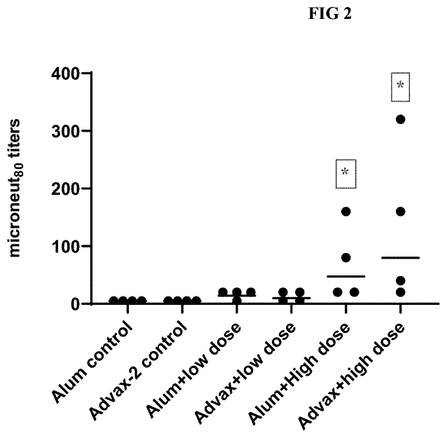

Microneutralization data (MN₈₀) from day 56 serum samples from mice (28 days after administering the second dose of vaccines) is shown in FIG. 2. At lower doses (10⁵ particles/dose) SARS-CoV-2 PsIV did not elicit any measurable neutralizing antibody response. However, at a higher dose (10⁷ particles/dose) SARS-CoV-2 PsIV elicited good neutralizing Ab response when compared to control animals. Interestingly, SARS-CoV-2 PsIV elicited a significantly higher neutralizing antibody response when formulated with advax-2 adjuvant, when compared to the response from SARS-CoV-2 formulated with the traditional alum adjuvant.

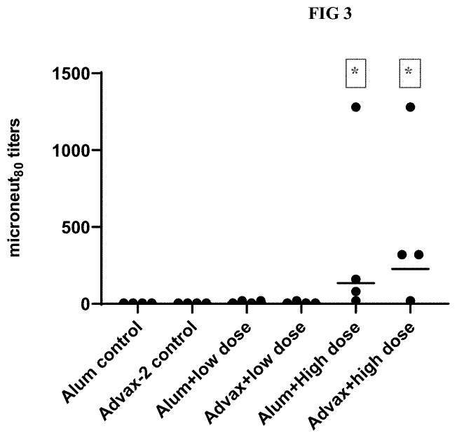

All animals were boosted on day 57 in preparation for harvesting the spleen cells on day 71 for measuring T-cell responses. Microneutralization data (MN₈₀) form day 71 serum samples are shown in FIG. 3. The high dose SARS-CoV-2 PsIV/Advax-2 vaccine group continued to exhibit higher neutralizing antibody responses when compared to the high dose SARS-CoV-2/alum vaccine group. Mice that received the low dose of SARS-CoV-2 PsIV did not show any neutralizing antibody responses, regardless of the adjuvant used. These results suggest that SARS-CoV-2 PsIV vaccine prepared according to this invention is capable of eliciting neutralizing antibody responses in animal models.

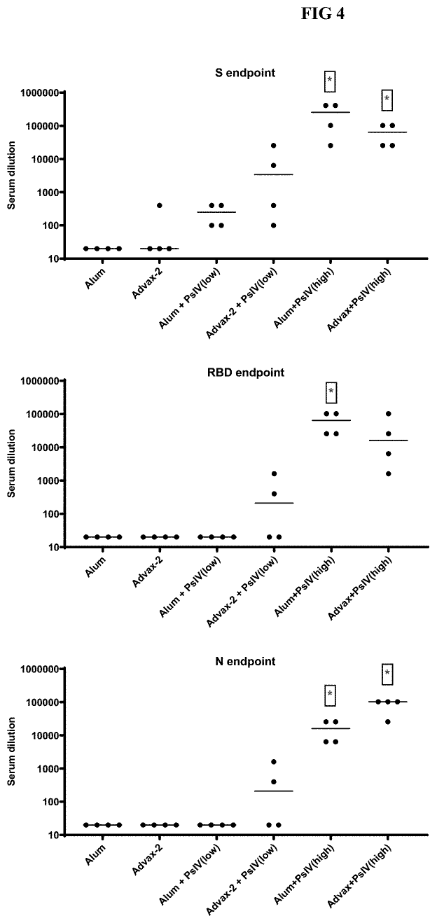

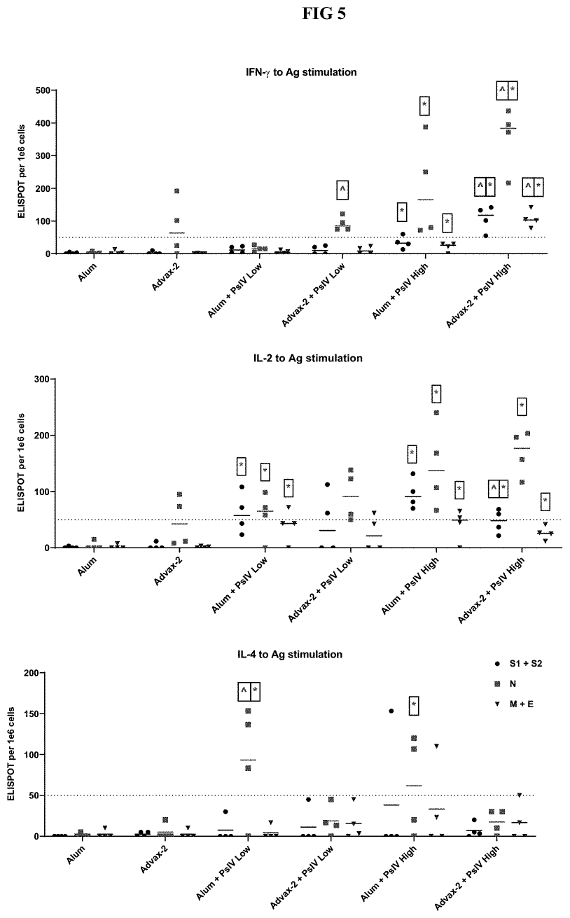

As illustrated in FIG. 4, antiSARS-CoV-2 IgG antibodies against the spike protein as well as the receptor binding domain of the spike protein were detected in day 71 sera obtained from mice vaccinated with SARS-CoV-2 PsIV. Anti-SARS-CoV-2 nucleocapsid protein IgG antibodies were also detected in day 71 sera samples from mice vaccinated with SARS-CoV-2 PsIV. Memory T-cell responses for different antigens were assessed by measuring the production of different cytokines. Interferon gamma (IFN-γ), interleukin 2 (IL-2), and interleukin 4 (IL-4) were measured by ELISPOT assays after stimulating the cells with three different SARS-CoV-2 antigen pools: S1+S2 peptides, N peptides and M+E peptides. As shown in FIG. 5, low dose of SARS-CoV-2 PsIV in alum induced a low IFN-γ response to N peptide pools, while a high dose of SARS-CoV-2 PsIV in alum induced a higher response to N peptide pools but not to the other two antigen pools. However a low dose of SARS-CoV-2 PsIV in advax-2 elicited a good IFN-γ response to N-antigens while a high dose of SARS-CoV-2 PsIV in advax-2 elicited a good IFN-γ responses to all three antigen pools. In general, SARS-CoV-2 PsIV in advax-2 elicited significantly higher IFN-y responses than the SARS-CoV-2 PsIV in alum. SARS-CoV-2 PsIV in alum but not SARS-CoV-2 PsIV in advax-2 elicited IL-4 responses. In general, cytokine responses after stimulation with different viral antigen peptide pools (such as spike protein and nucleocapsid protein) were observed for memory cells obtained from mice vaccinated with SARS-CoV-2 PsIV.

In summary, the results of our studies in mice and NHPs demonstrated that SARS-CoV-2 PsIV prepared according to this invention is capable of eliciting neutralizing antibodies and T-cell responses to SARS-CoV-2 spike and nucleocapsid proteins.

### REFERENCES

### 1. Cascella M, Rajnik M, Cuomo A, Dulebohn S C, and Di Napoli R, Features, Evaluation and Treatment Coronavirus (COVID-19), in StatPearls. 2020: Treasure Island (FL).

2. Walls A C, Park Y J, Tortorici MA , Wall A, McGuire A T, and Veesler D, Structure, Function, and Antigenicity of the SARS-CoV-2 Spike Glycoprotein. Cell, 2020.
3. Sep. 3, 2021]; WHO Coronavirus (COVID-19) dashboard]. Available from: https://covid19.who.int/.
4. Zhao J, Zhao S, Ou J, Zhang J, Lan W, Guan W, Wu X, Yan Y, Zhao W, Wu J, Chodosh J, and Zhang Q, COVID-19: Coronavirus Vaccine Development Updates. Front Immunol, 2020. 11: p. 602256.
5. Forni G, Mantovani A, and Covid-19 Commission of Accademia Nazionale dei Lincei R, COVID-19 vaccines: where we stand and challenges ahead. Cell Death Differ, 2021. 28(2): p. 626-639.
6. Chung J Y, Thone M N, and Kwon Y J, COVID-19 vaccines: The status and perspectives in delivery points of view. Adv Drug Deliv Rev, 2021. 170: p. 1-25.
7. Fraenkel-Conrat H and Mecham D K, The reaction of formaldehyde with proteins; demonstration of intermolecular cross-linking by means of osmotic pressure measurements. J Biol Chem, 1949. 177(1): p. 477-86.
8. Hotez P J and Bottazzi M E, Whole Inactivated Virus and Protein-Based COVID-19 Vaccines. Annu Rev Med, 2021.
9. Hotez P J, Nuzhath T, Callaghan T, and Colwell B, COVID-19 vaccine decisions: considering the choices and opportunities. Microbes Infect, 2021. 23(4-5): p. 104811.
10. Bonnafous P, Nicolai M C, Taveau J C, Chevalier M, Barriere F, Medina J, Le Bihan O, Adam O, Ronzon F, and Lambert O, Treatment of influenza virus with beta-propiolactone alters viral membrane fusion. Biochim Biophys Acta, 2014. 1838(1 Pt B): p. 355-63.
11. She Y M, Cheng K, Farnsworth A, Li X, and Cyr T D, Surface modifications of influenza proteins upon virus inactivation by beta-propiolactone. Proteomics, 2013. 13(23-24): p. 3537-47.
12. Wollowitz S, Fundamentals of the psoralen-based Helinx technology for inactivation of infectious pathogens and leukocytes in platelets and plasma. Semin Hematol, 2001. 38(4 Suppl 11): p. 4-11.
13. Hanson C V, Riggs J L, and Lennette E H, Photochemical inactivation of DNA and RNA viruses by psoralen derivatives. J Gen Virol, 1978. 40(2): p. 345-58.
14. Hanson C V, Photochemical inactivation of viruses with psoralens: an overview. Blood Cells, 1992. 18(1): p. 7-25.
15. Sundaram A K, Ewing D, Blevins M, Liang Z, Sink S, Lassan J, Raviprakash K, Defang G, Williams M, Porter K R, and Sanders J W, Comparison of purified psoralen-inactivated and formalin-inactivated dengue vaccines in mice and nonhuman primates. Vaccine, 2020. 38(17): p. 3313-3320.

What is claimed is:

1) A method to prepare a whole virus inactivated vaccine against coronavirus comprising:
a. adding one or more inactivating psoralen compound to said live coronavirus; and
b. exposing said coronavirus and psoralen compound mixture to a preselected intensity of an ultraviolet radiation for a preselected time long enough to render the virus non-replicative but short enough to prevent degradation of the virus's antigenic characteristics.

2) The method of claim 1, wherein said coronavirus is SARS-CoV-2, MERS-CoV, human coronavirus OC43, human coronavirus HKU1 or SARS-CoV.

3) The method of claim 2, wherein said SARS-CoV-2 is one of the SARS-CoV-2 variants of concern (VOC).

4) The method of claim 1, wherein said inactivating psoralen compound is added to a medium containing said live coronavirus.

5) The method of claim 4, wherein the said psoralen is selected from the group consisting of 4′-aminomethyltrioxsalen (AMT), 8-methoxy psoralen (8-MOP), 4.5′8-trimethylpsoralen (TMP), and a combination thereof.

6) The method of claim 4, wherein said psoralen is added to the medium at a concentration of 5 -150 m/mL.

7) The method claim 6, wherein the concentration of said psoralen is 10-50 μg/mL.

8) The method of claim 1, wherein the wavelength of said ultraviolet radiation is selected from 320-400 nm.

9) The method of claim 8, wherein the wavelength of said ultraviolet radiation is approximately 365 nm.

10) The method of claim 1, wherein the said ultraviolet radiation exposure is from 5-30 minutes.

11) The method of claim 10, wherein the said ultraviolet radiation exposure is 5-15 minutes.

12) The method of claim 1, wherein the said ultraviolet radiation intensity is 150 μW/cm² to 1500 μW/cm².

13) The method of claim 4, wherein SARS-CoV-2 virus concentration in the medium is 1×10⁵ to 1×10¹² pfu/mL.

14) The method of claim 1, wherein the temperature of said SARS-CoV-2 virus medium is maintained at 4° C. during said ultraviolet radiation exposure.

15) The method of claim 1, wherein said coronavirus and psoralen compound mixture is purified after said UV radiation exposure.

16) The method of claim 15, wherein said coronavirus and psoralen compound mixture is purified using sucrose gradient centrifugation or chromatography.

17) A psoralen inactivated whole virus vaccine against coronavirus, comprising one or more strains of inactivated coronavirus.

18) The psoralen inactivated whole virus vaccine of claim 17, wherein inactivated SARS-CoV-2 virus of one or more variants of concern

19) The psoralen inactivated whole virus vaccine of claim 17, wherein said vaccine comprise one or more variants of inactivated SARS-CoV-2 virus.

20) The psoralen inactivated whole virus vaccine of claim 17, wherein the said SARS-CoV-2 vaccine further comprises an adjuvant.

21) The psoralen inactivated whole virus vaccine of claim 20, wherein the said adjuvant is alum (alhydrogel), ASO4 (monophosphoryl lipid A), MF59 (oil-in-water emulsion containing squalene), CpG1018 or Advax-2.

22) A psoralen inactivated whole virus vaccine, wherein said vaccine is prepared by method of claims 1-16.

23) An immunogenic composition, comprising psoralen inactivated whole coronavirus and a pharmaceutical acceptable adjuvant.
